(19) United States
(12) Patent Application Publication
(10) Pub. No.: US 2006/0233892 A1
(43) Pub. Date: Oct. 19, 2006

(54) Topiramate compositions for treatment of headache

(76) Inventor: Curt Hendrix, Encino, CA (US)

Correspondence Address: KOPPEL, PATRICK & HEYBL, 555 ST. CHARLES DRIVE, SUITE 107, THOUSAND OAKS, CA, 91360, US

(21) Appl. No.: 11/406,953

(22) Filed: Apr. 18, 2006

Related U.S. Application Data
(60) Provisional application No. 60/673,099, filed on Apr. 19, 2005.
(60) Provisional application No. 60/674,107, filed on Apr. 21, 2005.

(51) Int. Cl. A61K 36/906; A61K 36/28; A61K 31/7024; A61K 31/525; A61K 31/205; A61K 31/185; A61K 31/35

(52) U.S. Cl. 424/702; 424/764; 424/756; 424/722; 514/23; 514/250; 514/460; 514/547; 514/560; 514/553; 514/562; 514/419; 514/554; 514/251

(57) ABSTRACT

A composition containing from about 10 to about 50 mg of topiramate in combination with one or more of parthenolide, magnesium and riboflavin is effective for treating headaches, and particularly migraine headaches, while reducing or eliminating the side effects experienced by users of prior higher dose topiramate compositions.

This application claims the benefit of U.S. Provisional Application No. 60/673,099 filed Apr. 19, 2005 and U.S. Provisional Application No. 60/674,107 filed Apr. 21, 2005.

### BACKGROUND

The drug topiramate, a sulfamate-substituted monosaccharide, designated chemically as 2,3:4,5-Di-O-isopropylidene-β-D-fructopyranose sulfamate or 2,3:4,5-bis-O-(l-methylethylidene-β-D-fructopyranose sulfamate, having the structural formula shown in FIG. 1, has been approved by the US Food and Drug Administration for prophylactic treatment of migraines. Multicentered, randomized, double-blind, placebo controlled, parallel group clinical trials of approximately 900 migraine sufferers, with open label studies on an additional 400 individuals, supporting this claim indicate that at 100 mg per day or greater, topiramate had statistically significant prophylactic effects versus placebo. However, there was no statistical significance (no substantial reduction in the reduction of migraine incidents at 50 mg/day or less of topiramate vs placebo. These studies also showed that patients receiving 100 mg or more per day of topiramate experienced significant unacceptable side effects including cognitive-related dysfunction (e.g. confusion, psychomotor slowing, difficulty with concentration/attention, memory loss, stupor, slurring of speech and word finding difficulty), depression and mood problems, somnolence, fatigue, parathesias, hyperventilation, anorexia, allergic reactions, chest pain, cardiac arrhythmias, liver malfinction, acute myopia, elevated ocular pressure, oligohidrosis and hyperthermia, among other symptoms. Additionally, more then 1% of the patients reported an increase in migraine and other headaches as well as other unacceptable side effects and in broader commercial use additional side effects were noted. While these side effects can be minimized or reduced to acceptable levels by lowering the daily dosage the studies also indicated that at 50 mg per day, toprimate did not show statistically significant efficacy for migraine prophylaxis versus placebo. Many patients when provided with the 100 mg dosage and the prospect of these serious side effects either refuse to take topiramate or will discontinue its use because of these side effects.

Ehrenberg, et al, U.S. Pat. Nos. 5,998,380 and 6,503,884 disclose the use of sulfamates, of which topiramate is one example, for treating migraines. The specific examples therein report on dosages of 600-800 mg/day in a first instance and 200-400 mg/d in a second instance of topiramate in order to obtain substantial improvement (reduction of migraine symptoms) but the dosage range was suggested as 50 mg-1000 mg per day which, according to the data before the FDA, includes ineffective dosages.

U.S. Pat. No. 6,319,903 describes the use of topiramate to treat cluster headaches. While the suggested dosage ranges from 15 mg to 1000 mg per day, preferably greater then 25 mg/day, the examples show that while eventually beneficial, all dosages (50, 100, or 125 mg/day) were inadequate in providing rapid relief and even with daily delivery in the 50-125 mg range it took 1-3 weeks to control the cluster headaches.

U.S. Pat. Nos. 6,559,293 and 6,699,840 disclose the use of salts of topiramate, preferably topiramate sodium, for the treatment of numerous physical conditions including neuropathic pain, migraine and cluster headaches. While dosages of 10 to 1500 mg are suggested, no data is provided regarding the relationship of the dosage to the efficacy of treatment

U.S. Pat. Nos. 6,500,450 and 6,068,999 issued to applicant and incorporated herein by reference disclose and claim the use of compositions containing a) parthenolide, particularly parthenolide extracted from feverefew or feverfew including naturally occurring parthenolide, b) riboflavin and c) magnesium provided by numerous different magnesium-containing chemical compositions, such as, magnesium in the form of acid salts, magnesium oxide, complexes or chelates. While these compositions have shown significant efficacy in treating and preventing migraines, they are not necessarily effective for all types of migraines or for other types of headaches.

### SUMMARY

A composition containing topiramate for treating headaches, and particularly migraine headaches, which is both effective and reduces or eliminates the side effects experienced by users of prior topiramate compositions is disclosed. The composition comprises a daily dosage of a combination of from about 10 to about 50 mg of topiramate in combination with one or more of parthenolide, magnesium and riboflavin. The composition provides more effective prevention and treatment of more types of headaches than topiramate or parthenolide, magnesium and riboflavin taken alone and allows the previously recommended dosage of the topiramate to be reduced while maintaining the beneficial clinical results, all with no, or significantly reduced unacceptable side effects experienced using larger dosages of topiramate.

### DESCRIPTION OF DRAWINGS

FIG. 1 shows the structural formula of topiramate.

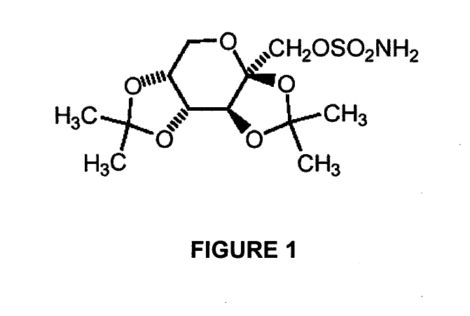

### DETAILED DESCRIPTION

Applicant has discovered that 10-75 mg per day, preferably 50 mg or less of topiramate, in combination with compositions comprising one or more of a) parthenolide, b) riboflavin and c) magnesium provided by numerous different magnesium-containing chemical compositions, such as, magnesium in the form of acid salts, magnesium oxide, complexes or chelates, provides patients with the same or better degree of prophylaxis of migraine, as well as other forms of headache, while significantly reducing the known side effects from topiramate. As used herein the term parthenolide includes, but is not limited to, parthenolide extracted from feverefew or other natural sources of parthenolide or the use of feverfew or other natural products including naturally occurring parthenolide, and synthetic parthenolides, chemically modified parthenolide or parthenolide derivates. The compositions are also more beneficial in treating a migraine headache should it occur. Preferred compositions comprise a combination including topiramate along with the ingredients and combination of ingredients set forth in U.S. Pat. Nos. 6,500,450 and 6,068,999. When 10-50 mg of topiramate is delivered in combination with one or more of parthenolide, magnesium and riboflavin as set forth herein it may be possible to also reduce the dosage of parthenolide, magnesium and riboflavin set forth in the '999 and '450 patent and still achieve equal or superior treatment of migraine, or other headaches, and reduction of the systemic symptoms commonly experienced by migraine suffers (nausea, sensitivity to light, aura, blurred or distorted vision, feelings of numbness in the body, throbbing sensations, etc.)

The compositions set forth herein will allow both physicians who treat chronic migraine patients and headache suffers, and the patients themselves, to effectively reduce the frequency of chronic migraine symptoms, reduce side effects and allow for continued use of this therapy because of increased tolerability for the active ingredients.

Preferred compositions for daily delivery, which may be provided in single or multiple dosages over a 24 hour period, comprise 10-50 mg or topiramate in combination with one or more of the following:
10-1000 mg of magnesium from any source, 10-1000 mg of riboflavin, and 10-1000 mg of the herb feverfew, either in the form of powdered whole herb, tinctures, extracts or other forms containing from about 0.1 to about 30 mg of parthenolide.

The preferred total dosage in a 24 hour period is 10-50 mg topiramate, and one or more of 200-400 mg riboflavin, 300-600 mg of magnesium and at least 0.2 mg of parthenolide, preferably delivered in two or three equal dosages spaced over that 24 hour period. The magnesium is preferably delivered as acid salts, complexes or chelates of magnesium and may also include magnesium oxide.

Added benefits can be obtained by also adding one or more of 10-500 mg of CoQ-10, 200 mcg-20 mg of any source of folates or folate metabolites or derivatives, 10-500 mg of vitamin B-6 or its derivatives or metabolites, 100 mcg to 5 mg of vitamin B-12 including but not limited to cyanobobalmin, hydroxcobalamin, methycobalamin, adenosylcobalamin, 10 mg to 10 grams of carnitine from various sources including 1-carnitine, acetyl carnitine, 250 mg to 10 grams of taurine, NMDA antagonists such as 10 m.g. to 3 grams acetylcysteine or 5 mg to 1,000 mg DL-2-aminophophonovaleric acid (APV), 100 mg to 3 grams carnosine, 50 mg to 4 grams Omega 3 essential fatty acids, 0.25 mg to 15 mg of Melatonin, 10 mg to 1000 mg of 5-HTP, 5 mg to 1000 mg 5-HT, 0.5 to 10 gm EPA or DHA, 500 mg-10 gr GABA, 100 mg-3 gr ginger, 100 mg-2.5 gr niacine, particularly as niacinamide or nicotinic acid, 100 mg-3 gr N-acetyl cysteine, 50 mcg to 5 mg of selenium, 100 mg-3 gr potassium and 100 mg to 5 grams of carnosine.

These compositions are not limited to use for treating or preventing migraine. They may be used for treating a broad range of headaches including but not limited to chronic daily headaches, cluster headaches and tension headaches which are not characterized as migraines.

I claim:

1. A composition for treating headaches, reducing the incidence of headaches, or reducing the severity of headaches comprising a daily dosage of a combination of from about 10 to about 50 mg of a sulfamate in combination with one or more of parthenolide, magnesium and riboflavin.

2. The composition of claim 1 wherein the sulfamate is topiramate or a salt thereof.

3. The composition of claims 1 wherein the one or more of parthenolide, magnesium and riboflavin are from about 10 to about 1000 mg of magnesium, from about 10 to about 1000 mg of riboflavin, and from about 0.1 to about 30 mg of parthenolide.

4. The composition of claim 3 wherein the parthenolide is provided by a) feverfew in the form of powder, tincture or extracts of feverfew or other naturally occurring sources of parthenolide, or b) synthetic parthenolide, chemically modified parthenolide or parthenolide derivatives.

5. The composition of claim 4 wherein from about 10 to about 1000 mg of feverfew powder is provided.

6. The composition of claim 1 further including one or more of CoQ-10, folates or folate metabolites or derivatives, vitamin B-6 or its derivatives or metabolites, vitamin B-12, carnitine, taurine, NMDA antagonists, DL-2-aminophophonovaleric acid, carnosine, Omega 3 essential fatty acids, melatonin, 5-HTP, 5-HT, EPA or DHA, GABA, ginger, niacine, N-acetyl cysteine, selenium, potassium or carnosine.

7. The composition of claims 1 further including one or more one or more of 10 to 500 mg of CoQ-10, 200 mcg to 20 mg of folates or folate metabolites or derivatives, 10 to 500 mg of vitamin B-6 or its derivatives or metabolites, 100 mcg to 5 mg of vitamin B-12, 10 mg to 10 grams of carnitine, 250 mg to 10 grams of taurine, 100 mg to 3 grams carnosine, 50 mg to 4 grams Omega 3 essential fatty acids, 0.25 mg to 15 mg of Melatonin, 10 mg to 1000 mg of 5-HTP, 5 mg to 1000 mg of 5-HT, 0.5 to 10 gm of EPA or DHA, 500 mg to 10 gr of GABA, 100 mg to 3 gr of ginger, 100 mg to 2.5 gr of niacine, 100 mg to 3 gr of N-acetyl cysteine, 50 mcg to 5 mg of selenium, 100 mg to 3 gr of potassium and 100 mg to 5 grams of carnosine.

8. The Composition of claim 7 wherein the vitamin B12 is in the form of cyanobobalmin, hydroxcobalamin, methycobalamin, adenosylcobalamin, carnitine is in the form of 1-carnitine or acetyl carnitine, the NMDA antagonists is 10 mg to 3 grams of acetylcysteine or 5 mg to 1,000 mg DL-2-aminophophonovaleric acid and the niacin, is in the form of niacinamide or nicotinic acid.

9. A composition for treating headaches, reducing the incidence of headaches, or reducing the severity of headaches comprising a daily dosage of a combination of from about 10 to about 50 mg of a topiramate in combination with one or more of 10 to about 1000 mg of magnesium, from about 10 to about 1000 mg of riboflavin, and from about 0.1 to about 30 mg of parthenolide.

10. The composition of claim 9 including from about of 300 to about 600 mg of magnesium, from about 200 to about 400 mg of riboflavin, and from greater than about 0.2 mg of parthenolide.

11. A composition for treating headaches comprising an effective daily dosage, prepared for oral delivery, of a combination of topiramate and one or more of magnesium, riboflavin, and parthenolide.
